(19) United States
(12) Patent Application Publication
(10) Pub. No.: US 2015/0165209 A1
(43) Pub. Date: Jun. 18, 2015

(54) SYSTEM AND METHOD FOR DELIVERING MODULATED SUB THRESHOLD THERAPY TO A PATIENT

(71) Applicant: Boston Scientific Neuromodulation Corporation, Valencia, CA (US)

(72) Inventors: Sarvani Grandhe, Valencia, CA (US); Sridhar Kothandaraman, Valencia, CA (US)

(21) Appl. No.: 14/571,526

(22) Filed: Dec. 16, 2014

Related U.S. Application Data
(60) Provisional application No. 61/917,275, filed on Dec. 17, 2013.

(51) Int. Cl. A61N 1/36; A61N 1/372

(52) U.S. Cl. CPC A61N 1/36164; A61N 1/37247

(57) ABSTRACT

A neuromodulation system configured for providing sub-threshold neuromodulation therapy to a patient. The neuromodulation system comprises a plurality of electrical terminals configured for being respectively coupled to a plurality of electrodes, modulation output circuitry configured for delivering modulation energy to the electrical terminals, a user interface for receiving input from a user, and control/processing circuitry configured for generating a super-threshold modulation program based on the received user-input, controlling the modulation output circuitry to deliver super-threshold modulation energy in accordance with the super-threshold modulation program, automatically deriving a plurality of different sub-threshold modulation programs from the super-threshold modulation program, and controlling the modulation output circuitry to deliver sub-threshold modulation energy to a patient in accordance with at least one of the sub-threshold modulation programs.

### CLAIM OF PRIORITY

This application claims the benefit of priority under 35 U.S.C. §119(e) of U.S. Provisional Patent Application Ser. No. 61/917,275, filed on Dec. 17, 2013, which is herein incorporated by reference in its entirety.

### FIELD OF THE INVENTION

The present inventions relate to tissue modulation systems, and more particularly, to programmable neuromodulation systems.

### BACKGROUND OF THE INVENTION

Implantable neuromodulation systems have proven therapeutic in a wide variety of diseases and disorders. Pacemakers and Implantable Cardiac Defibrillators (ICDs) have proven highly effective in the treatment of a number of cardiac conditions (e.g., arrhythmias). Spinal Cord Stimulation (SCS) systems have long been accepted as a therapeutic modality for the treatment of chronic pain syndromes, and the application of tissue stimulation has begun to expand to additional applications such as angina pectoralis and incontinence. Deep Brain Stimulation (DBS) has also been applied therapeutically for well over a decade for the treatment of refractory chronic pain syndromes, and DBS has also recently been applied in additional areas such as movement disorders and epilepsy. Further, in recent investigations, Peripheral Nerve Stimulation (PNS) systems have demonstrated efficacy in the treatment of chronic pain syndromes and incontinence, and a number of additional applications are currently under investigation. Furthermore, Functional Electrical Stimulation (FES) systems, such as the Freehand system by NeuroControl (Cleveland, Ohio), have been applied to restore some functionality to paralyzed extremities in spinal cord injury patients.

These implantable neuromodulation systems typically include one or more electrode carrying stimulation leads, which are implanted at the desired stimulation site, and an implantable neuromodulation device (e.g., an implantable pulse generator (IPG)) implanted remotely from the stimulation site, but coupled either directly to the neuromodulation lead(s) or indirectly to the neuromodulation lead(s) via a lead extension. The neuromodulation system may further comprise a handheld external control device (e.g., a remote control (RC)) to remotely instruct the neuromodulator to generate electrical stimulation pulses in accordance with selected stimulation parameters.

Electrical modulation energy may be delivered from the neuromodulation device to the electrodes in the form of an electrical pulsed waveform. Thus, electrical energy may be controllably delivered to the electrodes to therapeutically modulate neural tissue. The configuration of electrodes used to deliver electrical pulses to the targeted tissue constitutes an electrode configuration, with the electrodes capable of being selectively programmed to act as anodes (positive), cathodes (negative), or left off (zero). In other words, an electrode configuration represents the polarity being positive, negative, or zero. Other parameters that may be controlled or varied include the amplitude, width, and rate of the electrical pulses (which may be considered electrical pulse parameters) provided through the electrode array. Each electrode configuration, along with the electrical pulse parameters, can be referred to as a “modulation parameter set.”

With some neuromodulation systems, and in particular, those with independently controlled current or voltage sources, the distribution of the current to the electrodes (including the case of the neuromodulation device, which may act as an electrode) may be varied such that the current is supplied via numerous different electrode configurations. In different configurations, the electrodes may provide current or voltage in different relative percentages of positive and negative current or voltage to create different electrical current distributions (i.e., fractionalized electrode configurations).

As briefly discussed above, an external control device can be used to instruct the neuromodulation device to generate electrical pulses in accordance with the selected modulation parameters. Typically, the modulation parameters programmed into the neuromodulation device can be adjusted by manipulating controls on the handheld external control device to modify the electrical modulation energy provided by the neuromodulation device system to the patient. Thus, in accordance with the modulation parameters programmed by the external control device, electrical pulses can be delivered from the neuromodulation device to the electrode(s) to modulate a volume of tissue in accordance with a set of modulation parameters and provide the desired efficacious therapy to the patient. The best modulation set will typically be one that delivers modulation energy to the volume of tissue that must be modulated in order to provide the therapeutic benefit (e.g., treatment of pain), while minimizing the volume of non-target tissue that is modulated.

However, the number of electrodes available combined with the ability to generate a variety of complex electrical pulses, presents a huge selection of modulation parameter sets to the clinician or patient. For example, if the neuromodulation system to be programmed has an array of sixteen electrodes, millions of modulation parameter sets may be available for programming into the neuromodulation system. Today, neuromodulation systems may have up to thirty-two electrodes, thereby exponentially increasing the number of modulation parameters sets available for programming.

To facilitate such selection, the clinician generally programs the neuromodulation device through a computerized programming system. This programming system can be a self-contained hardware/software system, or can be defined predominantly by software running on a standard personal computer (PC). The PC or custom hardware may actively control the characteristics of the electrical stimulation generated by the neuromodulation device to allow the optimum stimulation parameters to be determined based on patient feedback or other means and to subsequently program the neuromodulation device with the optimum modulation parameter sets.

For example, in order to achieve an effective result from conventional SCS, the lead or leads must be placed in a location, such that the electrical modulation energy (in this case, electrical stimulation energy) creates a sensation known as paresthesia, which can be characterized as an alternative sensation that replaces the pain signals sensed by the patient. The paresthesia induced by the stimulation and perceived by the patient should be located in approximately the same place in the patient's body as the pain that is the target of treatment. If a lead is not correctly positioned, it is possible that the patient will receive little or no benefit from an implanted SCS system. Thus, correct lead placement can mean the difference between effective and ineffective pain therapy. When electrical leads are implanted within the patient, the computerized programming system, in the context of an operating room (OR) mapping procedure, may be used to instruct the neuromodulation device to apply electrical stimulation to test placement of the leads and/or electrodes, thereby assuring that the leads and/or electrodes are implanted in effective locations within the patient.

Once the leads are correctly positioned, a fitting procedure, which may be referred to as a navigation session, may be performed using the computerized programming system to program the external control device, and if applicable the neuromodulation device, with a set of modulation parameters that best addresses the painful site. Thus, the navigation session may be used to pinpoint volume of activation (VOA) or areas correlating to the pain. Such programming ability is particularly advantageous for targeting the tissue during implantation, or after implantation should the leads gradually or unexpectedly move that would otherwise relocate the stimulation energy away from the target site. By reprogramming the neuromodulation device (typically by independently varying the stimulation energy on the electrodes), the volume of activation (VOA) can often be moved back to the effective pain site without having to re-operate on the patient in order to reposition the lead and its electrode array. When adjusting the volume of activation (VOA) relative to the tissue, it is desirable to make small changes in the proportions of current, so that changes in the spatial recruitment of nerve fibers will be perceived by the patient as being smooth and continuous and to have incremental targeting capability.

Although alternative or artifactual sensations are usually tolerated relative to the sensation of pain, patients sometimes report these sensations to be uncomfortable, and therefore, they can be considered an adverse side-effect to neuromodulation therapy in some cases. Because the perception of paresthesia has been used as an indicator that the applied electrical energy is, in fact, alleviating the pain experienced by the patient, the amplitude of the applied electrical energy is generally adjusted to a level that causes the perception of paresthesia. It has been shown, however, that the delivery of sub-threshold electrical energy (e.g., high frequency pulsed electrical energy and/or low pulse width electrical energy) can be effective in providing neuromodulation therapy for chronic pain without causing paresthesia.

Although sub-threshold modulation therapies have shown good efficacy in early studies, because there is a lack of paresthesia that may otherwise indicate that the delivered sub-threshold electrical energy is optimized, or at least efficacious, it is difficult to immediately determine if the delivered sub-threshold therapy is optimized in terms of providing efficacious therapy. Because the patient is unable to provide immediate feedback due to the lack of paresthesia, the clinician may program the IPG with various combinations of sub-threshold modulation programs to be tested on the patient until the next programming session. To this end, if the clinician believes that a first sub-threshold modulation program is efficacious for the patient, the clinician may want to try similar combinations of sub-threshold modulation programs based on the first sub-threshold modulation program to find an optimal treatment plan for sub-threshold therapy. Given the innumerable combinations of sub-threshold modulation programs, manually creating and testing out these various similar combinations of sub-threshold modulation programs is time-consuming and inefficient, often taking several days, if not weeks, and typically requires several reprogramming sessions with the clinician.

There, thus, remains a need to provide a more efficient means to program a neuromodulation system with sub-threshold modulation programs.

### SUMMARY OF THE INVENTION

In accordance with a first aspect of the present inventions, a method of providing sub-threshold modulation therapy to a patient comprises receiving input from a user, generating a super-threshold modulation therapy based on the received user-input, delivering super-threshold modulation energy to the patient in accordance with the super-threshold modulation program, automatically deriving a plurality of different sub-threshold modulation programs from the super-threshold modulation program, storing the plurality of sub-threshold modulation programs in memory, and delivering sub-threshold modulation energy (e.g., pulse width less than 100 μs, pulse rate greater than 1500 Hz, etc.) to the patient in accordance with at least one of the sub-threshold modulation programs, thereby providing therapy to the patient. The super-threshold modulation programs and the sub-threshold modulation programs comprise a plurality of modulation parameter sets respectively corresponding to different areas of the patient. Each of the plurality of sub-threshold modulation programs defines an inter-burst quiescent period of at least 1 ms and less than 5 seconds, and an intra-burst interval in the range of 0.1 ms to 10 ms.

The method further comprises receiving additional input from the user, selecting, based on the additional user-input, one of the plurality of sub-threshold modulation programs, determining a perception threshold of the selected sub-threshold modulation program, determining a sub-threshold amplitude value for the selected sub-threshold modulation program as a function (e.g., percentage) of the perception threshold to calibrate the selected sub-threshold modulation program, and storing the calibrated sub-threshold modulation program in the memory. The percentage is typically in the range of 30%-70%. In an optional embodiment, the calibrated sub-threshold modulation program may be saved onto a remote programming device. The user may define a minimum amplitude level and a maximum amplitude level for the saved sub-threshold modulation program to be used from the remote programming device.

The method further comprises repeatedly cycling through the plurality of sub-threshold modulation programs to deliver the sub-threshold modulation energy to the patient.

The method may further comprise receiving additional input from the user, and deleting one of the sub-threshold modulation programs from the memory in response to the additional user-input. The method further comprises automatically deriving another sub-threshold modulation program from another selected super-threshold modulation program, and storing the other sub-threshold modulation program in memory in place of the deleted sub-threshold modulation program.

In accordance with a second aspect of the present inventions, a neuromodulation system comprises a plurality of electrical terminals configured for being respectively coupled to a plurality of electrodes, modulation output circuitry configured for delivering modulation energy to the electrical terminals, a user interface for receiving input from a user, and control/processing circuitry configured for generating a super-threshold modulation program based on the received user-input, controlling the modulation output circuitry to deliver super-threshold modulation energy in accordance with the super-threshold modulation program, automatically deriving a plurality of different sub-threshold modulation programs from the super-threshold modulation program, and controlling the modulation output circuitry to deliver sub-threshold modulation energy (e.g., pulse width less than 100 μs, pulse rate greater than 1500 Hz, etc.) to a patient in accordance with at least one of the sub-threshold modulation programs.

The neuromodulation system further comprises memory configured for storing at least one of the super-threshold modulation program and the plurality of sub-threshold modulation programs. The super-threshold modulation program and the plurality of sub-threshold modulation programs comprise a plurality of modulation parameter sets respectively corresponding to different areas of the patient. Each of the plurality of sub-threshold modulation programs defines an inter-burst quiescent period of at least 1 ms and less than 5 seconds, and an intra-burst interval in the range of 0.1 ms to 10 ms.

The control/processing circuitry may be further configured for selecting, based on an additional user-input, one of the plurality of sub-threshold modulation programs, controlling the modulation output circuitry in a manner such that an amplitude value of the electrical energy delivered in accordance with the selected sub-threshold modulation program is incrementally adjusted. The user interface may be further configured for receiving further additional input from the user when the patient indicates perceiving paresthesia in response to the incrementally adjusted amplitude value. The control/processing circuitry may be configured for recording the adjusted amplitude value as a perception threshold based on the received further additional user-input.

The control/processing circuitry may be further configured for calculating a sub-threshold amplitude value for the selected sub-threshold modulation program as a function (e.g., percentage) of the perception threshold to calibrate the selected sub-threshold modulation program, and storing the calibrated sub-threshold modulation program in the memory. The percentage is typically in the range of 30% to 70%.

In an optional embodiment, the neuromodulation system further comprises a remote programming device having another user interface configured for receiving input from the patient and remotely controlling the modulation output circuitry. The control/processing circuitry is further configured for saving the calibrated sub-threshold modulation program onto the remote programming device.

The control/processing circuitry is further configured for defining a minimum amplitude level and a maximum amplitude level for the saved sub-threshold modulation program based on the received more further additional user-input. The control/processing circuitry is further configured for controlling the modulation output circuitry in a manner such that the plurality of sub-threshold modulation programs are repeatedly cycled.

The control/processing is further configured for deleting a sub-threshold modulation program based on a received additional user-input, generating another super-threshold modulation program based on a received further additional user-input, controlling the modulation output circuitry to deliver super-threshold modulation energy in accordance with the other super-threshold modulation program, automatically deriving another sub-threshold modulation program from the other super-threshold modulation program, and storing the other sub-threshold modulation program in memory in place of the deleted sub-threshold modulation program.

In accordance with a third aspect of the present inventions, an external control device for programming an implantable neuromodulator coupled to an electrode array comprises a user interface including control elements for receiving input from a user, telemetry circuitry configured for communicating with the neuromodulator, and control/processing circuitry configured for generating a super-threshold modulation program based on the received user-input, directing the neuromodulator to deliver super-threshold modulation energy in accordance with the super-threshold modulation program, automatically deriving a plurality of different sub-threshold modulation programs from the super-threshold modulation program, and directing the neuromodulator to deliver sub-threshold modulation energy (e.g., pulse width less than 100 μs, pulse rate greater than 1500 Hz, etc.) to a patient in accordance with at least one of the sub-threshold modulation programs.

The control/processing circuitry is further configured for calibrating the sub-threshold modulation program in the same manner described above. The control/processing circuitry is configured for deleting a sub-threshold modulation program, and generating another sub-threshold modulation program in place of the deleted sub-threshold modulation program in the same manner described above. The control/processing circuitry is further configured for directing the neuromodulator in a manner such that the plurality of sub-threshold modulation programs are repeatedly cycled in the same manner described above.

In an optional embodiment, the control/processing circuitry is further configured for communicating with a remote programming device for the same functions described above.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a plan view of a Spinal Cord Modulation (SCM) system constructed in accordance with one embodiment of the present inventions;

FIG. 2 is a plan view of the SCM system of FIG. 1 in use with a patient;

FIG. 3 is a profile view of an implantable pulse generator (IPG) and percutaneous leads used in the SCM system of FIG. 1;

FIG. 4 is a plot of monophasic cathodic electrical modulation energy;

FIG. 5a is a plot of biphasic electrical modulation energy having a cathodic modulation pulse and an active charge recovery pulse;

FIG. 5b is a plot of biphasic electrical modulation energy having a cathodic modulation pulse and a passive charge recovery pulse;

FIG. 6 is a block diagram of a clinician's programmer (CP) used in the SCM system of FIG. 1;

FIG. 7 is a plan view of a user interface of the CP of FIG. 6 for programming the IPG of FIG. 3 in a manual programming mode;

FIG. 8 is a plan view of a user interface of the CP of FIG. 6 illustrating a sub-threshold modulation program screen, wherein a plurality of sub-threshold modulation programs is automatically generated;

FIG. 9 is a plan view of a user interface of the CP of FIG. 6 illustrating a sub-threshold modulation program screen, wherein the automatically generated sub-threshold modulation program of FIG. 8 is calibrated; and

FIG. 10 is a plan view of a user interface of the CP of FIG. 6 illustrating a sub-threshold modulation program screen, wherein the automatically generated sub-threshold modulation program of FIG. 8 is deleted.

### DETAILED DESCRIPTION OF THE EMBODIMENTS

The description that follows relates to a spinal cord modulation (SCM) system. However, it is to be understood that the while the invention lends itself well to applications in SCM, the invention, in its broadest aspects, may not be so limited. Rather, the invention may be used with any type of implantable electrical circuitry used to stimulate tissue. For example, the present invention may be used as part of a pacemaker, a defibrillator, a cochlear stimulator, a retinal stimulator, a stimulator configured to produce coordinated limb movement, a cortical stimulator, a deep brain stimulator, peripheral nerve stimulator, microstimulator, or in any other neural stimulator configured to treat urinary incontinence, sleep apnea, shoulder sublaxation, headache, etc.

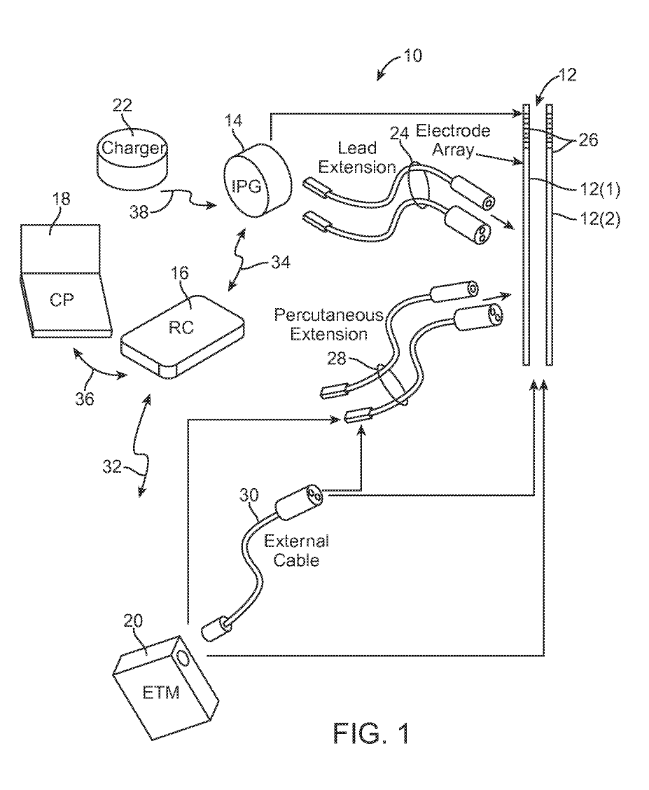

Turning first to FIG. 1, an exemplary SCM system 10 generally includes a plurality (in this case, two) of implantable neuromodulation leads 12, an implantable pulse generator (IPG) 14, an external remote controller RC 16, a clinician's programmer (CP) 18, an external trial modulator (ETM) 20, and an external charger 22.

The IPG 14 is physically connected via one or more percutaneous lead extensions 24 to the neuromodulation leads 12, which carry a plurality of electrodes 26 arranged in an array. In the illustrated embodiment, the neuromodulation leads 12 are percutaneous leads, and to this end, the electrodes 26 are arranged in-line along the neuromodulation leads 12. The number of neuromodulation leads 12 illustrated is two, although any suitable number of neuromodulation leads 12 can be provided, including only one. Alternatively, a surgical paddle lead can be used in place of one or more of the percutaneous leads. As will be described in further detail below, the IPG 14 includes pulse generation circuitry that delivers electrical modulation energy in the form of a pulsed electrical waveform (i.e., a temporal series of electrical pulses) to the electrode array 26 in accordance with a set of modulation parameters.

The ETM 20 may also be physically connected via the percutaneous lead extensions 28 and external cable 30 to the neuromodulation leads 12. The ETM 20, which has similar pulse generation circuitry as the IPG 14, also delivers electrical modulation energy in the form of a pulse electrical waveform to the electrode array 26 accordance with a set of modulation parameters. The major difference between the ETM 20 and the IPG 14 is that the ETM 20 is a non-implantable device that is used on a trial basis after the neuromodulation leads 12 have been implanted and prior to implantation of the IPG 14, to test the responsiveness of the modulation that is to be provided. Thus, any functions described herein with respect to the IPG 14 can likewise be performed with respect to the ETM 20. For purposes of brevity, the details of the ETM 20 will not be described herein.

The RC 16 may be used to telemetrically control the ETM 20 via a bi-directional RF communications link 32. Once the IPG 14 and neuromodulation leads 12 are implanted, the RC 16 may be used to telemetrically control the IPG 14 via a bi-directional RF communications link 34. Such control allows the IPG 14 to be turned on or off and to be programmed with different modulation parameter sets. The IPG 14 may also be operated to modify the programmed modulation parameters to actively control the characteristics of the electrical modulation energy output by the IPG 14. As will be described in further detail below, the CP 18 provides clinician detailed modulation parameters for programming the IPG 14 and ETM 20 in the operating room and in follow-up sessions.

The CP 18 may perform this function by indirectly communicating with the IPG 14 or ETM 20, through the RC 16, via an IR communications link 36. Alternatively, the CP 18 may directly communicate with the IPG 14 or ETM 20 via an RF communications link (not shown). The clinician detailed modulation parameters provided by the CP 18 are also used to program the RC 16, so that the modulation parameters can be subsequently modified by operation of the RC 16 in a stand-alone mode (i.e., without the assistance of the CP 18).

The external charger 22 is a portable device used to transcutaneously charge the IPG 14 via an inductive link 38. Once the IPG 14 has been programmed, and its power source has been charged by the external charger 22 or otherwise replenished, the IPG 14 may function as programmed without the RC 16 or CP 18 being present. For purposes of brevity, the details of the external charger 22 will not be described herein.

For purposes of brevity, the details of the CP 18, ETM 20, and external charger 22 will not be described herein. Details of exemplary embodiments of these devices are disclosed in U.S. Pat. No. 6,895,280, which is expressly incorporated herein by reference.

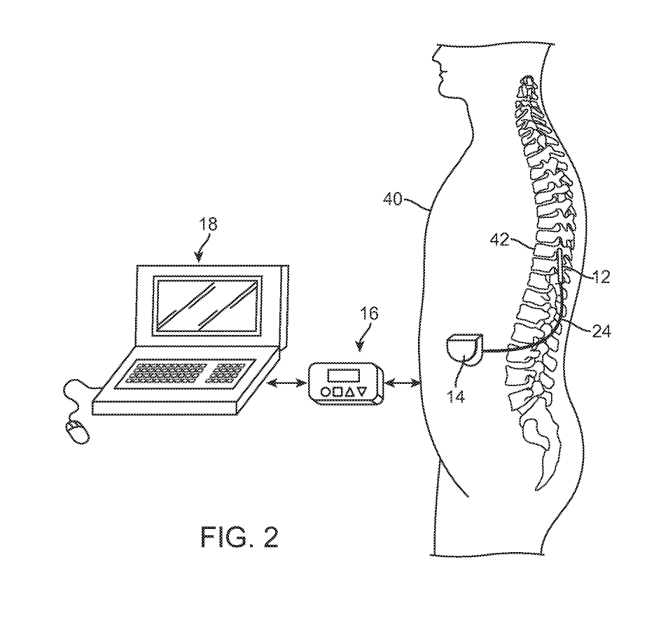

As shown in FIG. 2, the neuromodulation leads 12 are implanted within the spinal column 42 of a patient 40. The preferred placement of the neuromodulation leads 12 is adjacent, i.e., resting upon, the spinal cord area to be stimulated. Due to the lack of space near the location where the neuromodulation leads 12 exit the spinal column 42, the IPG 14 is generally implanted in a surgically-made pocket either in the abdomen or above the buttocks. The IPG 14 may, of course, also be implanted in other locations of the patient's body. The lead extension 24 facilitates locating the IPG 14 away from the exit point of the neuromodulation leads 12. As there shown, the CP 18 communicates with the IPG 14 via the RC 16.

The IPG 14 comprises an outer case 40 for housing the electronic and other components (described in further detail below), and a connector 42 to which the proximal ends of the neuromodulation leads 12 mate in a manner that electrically couples the electrodes 26 to the electronics within the outer case 40. The outer case 40 is composed of an electrically conductive, biocompatible material, such as titanium, and forms a hermetically sealed compartment wherein the internal electronics are protected from the body tissue and fluids. In some cases, the outer case 40 may serve as an electrode.

The IPG 14 includes a pulse generation circuitry that provides electrical modulation energy to the electrodes 26 in accordance with a set of modulation parameters. Such parameters may include electrode combinations, which define the electrodes that are activated as anodes (positive), cathodes (negative), and turned off (zero). The modulation parameters may further include pulse amplitude (measured in milliamps or volts depending on whether the IPG 14 supplies constant current or constant voltage to the electrodes), pulse width (measured in microseconds), pulse rate (measured in pulses per second), duty cycle (pulse width divided by cycle duration), burst rate (measured as the modulation energy on duration X and modulation energy off duration Y), and pulse shape.

With respect to the pulse patterns provided during operation of the system 10, electrodes that are selected to transmit or receive electrical energy are referred to herein as “activated,” while electrodes that are not selected to transmit or receive electrical energy are referred to herein as “non-activated.” Electrical energy delivery will occur between two (or more) electrodes, one of which may be the IPG outer case 40. Electrical energy may be transmitted to the tissue in a monopolar or multipolar (for example, bipolar, tripolar and similar configurations) fashion or by any other means available.

The IPG 14 may be operated in either a super-threshold delivery mode or a sub-threshold delivery mode. While in the super-threshold delivery mode, the IPG 14 is configured for delivering electrical modulation energy that provides super-threshold therapy to the patient (in this case, causes the patient to perceive paresthesia). For example, an exemplary super-threshold pulse train may be delivered at a relatively high pulse amplitude (e.g., 5 ma), a relatively low pulse rate (e.g., less than 1500 Hz, preferably less than 500 Hz), and a relatively high pulse width (e.g., greater than 100 μs, preferably greater than 200 μs).

While in the sub-threshold delivery mode, the IPG 14 is configured for delivering electrical modulation energy that provides sub-threshold therapy to the patient (in this case, does not cause the patient to perceive paresthesia). For example, an exemplary sub-threshold pulse train may be delivered at a relatively low pulse amplitude (e.g., 2.5 ma), a relatively high pulse rate (e.g., greater than 1500 Hz, preferably greater than 2500 Hz), and a relatively low pulse width (e.g., less than 100 μs, preferably less than 50 μs).

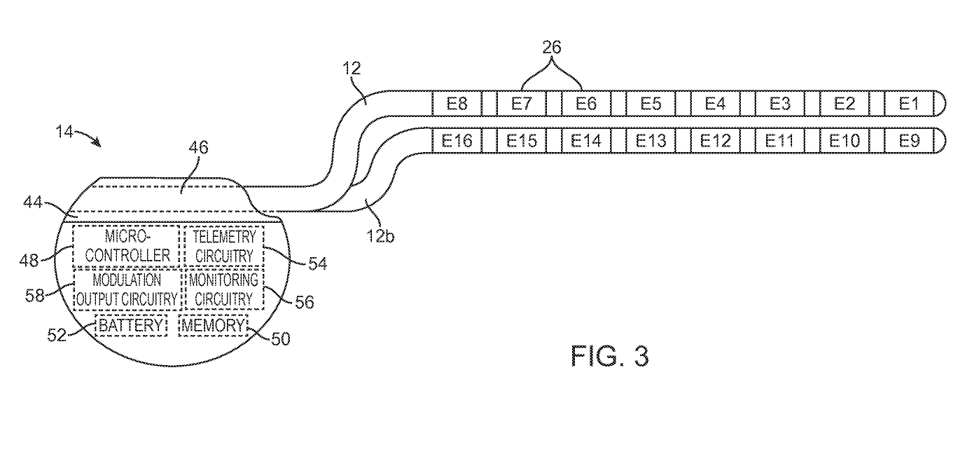

Referring now to FIG. 3, the external features of the neuromodulation leads 12 and the IPG 14 will be briefly described. One of the neuromodulation leads 12a has eight electrodes 26 (labeled E1-E8), and the other neuromodulation lead 12b has eight electrodes 26 (labeled E9-E16). The actual number and shape of leads and electrodes will, of course, vary according to the intended application. The IPG 14 comprises an outer case 44 for housing the electronic and other components (described in further detail below), and a connector 46 to which the proximal ends of the neuromodulation leads 12 mates in a manner that electrically couples the electrodes 26 to the electronics within the outer case 44. The outer case 44 is composed of an electrically conductive, biocompatible material, such as titanium, and forms a hermetically sealed compartment wherein the internal electronics are protected from the body tissue and fluids. In some cases, the outer case 44 may serve as an electrode.

The IPG 14 comprises electronic components, such as a controller/processor (e.g., a microcontroller) 48, memory 50, a battery 52, telemetry circuitry 54, monitoring circuitry 56, modulation output circuitry 58, and other suitable components known to those skilled in the art. The microcontroller 48 executes a suitable program stored in memory 50, for directing and controlling the neuromodulation performed by IPG 14. Telemetry circuitry 54, including an antenna (not shown), is configured for receiving programming data (e.g., the operating program and/or modulation parameters) from the RC 16 and/or CP 18 in an appropriate modulated carrier signal, which the programming data is then stored in the memory (not shown). The telemetry circuitry 54 is also configured for transmitting status data to the RC 16 and/or CP 18 in an appropriate modulated carrier signal. The battery 52, which may be a rechargeable lithium-ion or lithium-ion polymer battery, provides operating power to IPG 14. The monitoring circuitry 56 is configured for monitoring the present capacity level of the battery 43.

The modulation output circuitry 58 provides electrical modulation energy in the form of a pulsed electrical waveform to the electrodes 26 in accordance with a set of modulation parameters programmed into the IPG 14. Such modulation parameters may comprise electrode combinations, which define the electrodes that are activated as anodes (positive), cathodes (negative), and turned off (zero), percentage of modulation energy assigned to each electrode (fractionalized electrode configurations), and electrical pulse parameters, which define the pulse amplitude (measured in milliamps or volts depending on whether the IPG 14 supplies constant current or constant voltage to the electrode array 26), pulse width (measured in microseconds), pulse rate (measured in pulses per second), and burst rate (measured as the modulation on duration X and modulation off duration Y).

Electrical modulation will occur between two (or more) activated electrodes, one of which may be the IPG case 44. Modulation energy may be transmitted to the tissue in a monopolar or multipolar (e.g., bipolar, tripolar, etc.) fashion. Monopolar modulation occurs when a selected one of the lead electrodes 26 is activated along with the case of the IPG 14, so that modulation energy is transmitted between the selected electrode 26 and case. Bipolar modulation occurs when two of the lead electrodes 26 are activated as anode and cathode, so that modulation energy is transmitted between the selected electrodes 26. For example, electrode E3 on the first lead 12a may be activated as an anode at the same time that electrode E11 on the second lead 12b is activated as a cathode. Tripolar modulation occurs when three of the lead electrodes 26 are activated, two as anodes and the remaining one as a cathode, or two as cathodes and the remaining one as an anode. For example, electrodes E4 and E5 on the first lead 12a may be activated as anodes at the same time that electrode E12 on the second lead 12b is activated as a cathode.

Any of the electrodes E1-E16 and case electrode may be assigned to up to k possible groups or timing “channels.” In one embodiment, k may equal four. The timing channel identifies which electrodes are selected to synchronously source or sink current to create an electric field in the tissue to be stimulated. Amplitudes and polarities of electrodes on a channel may vary. In particular, the electrodes can be selected to be positive (sourcing current), negative (sinking current), or off (no current) polarity in any of the k timing channels.

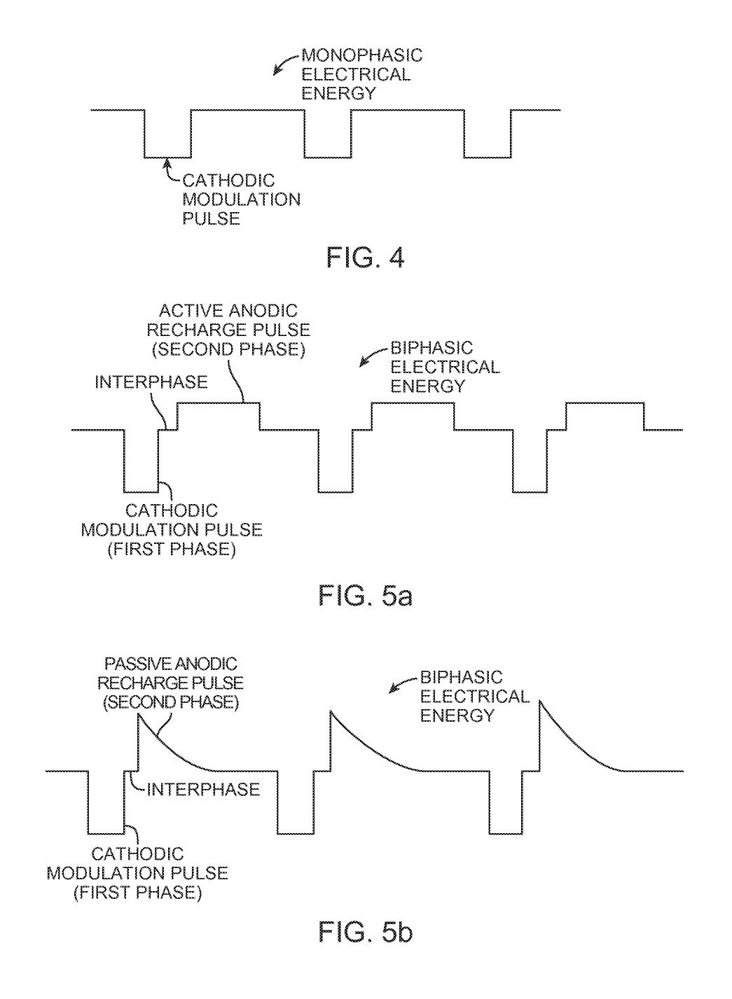

The modulation energy may be delivered between a specified group of electrodes as monophasic electrical energy or multiphasic electrical energy. As illustrated in FIG. 4, monophasic electrical energy takes the form of an electrical pulse train that includes either all negative pulses (cathodic), or alternatively all positive pulses (anodic).

Multiphasic electrical energy includes a series of pulses that alternate between positive and negative. For example, as illustrated in FIGS. 5a and 5b, multiphasic electrical energy may include a series of biphasic pulses, with each biphasic pulse including a cathodic (negative) modulation phase and an anodic (positive) charge recovery pulse phase that is generated after the modulation phase to prevent direct current charge transfer through the tissue, thereby avoiding electrode degradation and cell trauma. That is, charge is conveyed through the electrode-tissue interface via current at an electrode during a modulation period (the length of the modulation phase), and then pulled back off the electrode-tissue interface via an oppositely polarized current at the same electrode during a recharge period (the length of the charge recovery phase).

The second phase may be an active charge recovery phase (FIG. 5a), wherein electrical current is actively conveyed through the electrode via current or voltage sources, or the second phase may be a passive charge recovery phase (FIG. 5b), wherein electrical current is passively conveyed through the electrode via redistribution of the charge flowing from coupling capacitances present in the circuit. Using active recharge, as opposed to passive recharge, allows faster recharge, while avoiding the charge imbalance that could otherwise occur. Another electrical pulse parameter in the form of an interphase can define the time period between the pulses of the biphasic pulse (measured in microseconds). Although the modulation and charge recovery phases of the biphasic pulses illustrated in FIGS. 5a and 5b are cathodic and anodic, respectively, it should be appreciated that the modulation and charge recovery pulses of biphasic pulses may be anodic and cathodic, respectively, depending upon the desired therapeutic result.

In the illustrated embodiment, IPG 14 can individually control the magnitude of electrical current flowing through each of the electrodes. In this case, it is preferred to have a current generator, wherein individual current-regulated amplitudes from independent current sources for each electrode may be selectively generated. Although this system is optimal to take advantage of the invention, other neuromodulators that may be used with the invention include neuromodulators having voltage regulated outputs. While individually programmable electrode amplitudes are optimal to achieve fine control, a single output source switched across electrodes may also be used, although with less fine control in programming. Mixed current and voltage regulated devices may also be used with the invention. Further details discussing the detailed structure and function of IPGs are described more fully in U.S. Pat. Nos. 6,516,227 and 6,993,384, which are expressly incorporated herein by reference.

It should be noted that rather than an IPG, the SCM system 10 may alternatively utilize an implantable receiver-stimulator (not shown) connected to the neuromodulation leads 12. In this case, the power source, e.g., a battery, for powering the implanted receiver, as well as control circuitry to command the receiver-stimulator, will be contained in an external controller inductively coupled to the receiver-stimulator via an electromagnetic link. Data/power signals are transcutaneously coupled from a cable-connected transmission coil placed over the implanted receiver-modulator. The implanted receiver-modulator receives the signal and generates the modulation in accordance with the control signals.

More significant to the present inventions, since, due to the lack of paresthesia, it is especially difficult to find an appropriate modulation program in the case of sub-threshold modulation therapy, the SCM system 10 is configured for automatically generating a plurality of sub-threshold modulation programs based on a super-threshold modulation program that is believed to be effective. Thus, instead of manually selecting and trying out various combinations and permutations of sub-threshold modulation programs in an effort to find an optimal set of modulation parameters, the clinician may select and test the automatically generated sub-threshold modulation programs, thereby making the process more efficient and time-effective.

In particular, when evaluating the patient's needs and targeted area for neuromodulation therapy, the user may identify a particular super-threshold modulation program that may work for the patient (e.g., the patient may feel paresthesia in the targeted therapy area in response to the super-threshold modulation program). Based on this effective super-threshold modulation program, the SCM system 10 automatically generates a plurality (e.g., five, eight, ten, etc.) of sub-threshold modulation programs that is similar to the selected super-threshold modulation program.

In creating the plurality of sub-threshold modulation programs based on the selected super-threshold modulation program, the SCM system 10 may slightly modify existing modulation parameters or generate new combinations that are somewhat similar to the existing modulation parameters while maintaining the electrical pulse train of the sub-threshold modulation program at a sub-threshold level (i.e., pulse width less than 100 μs and/or pulse rate greater than 1500 Hz). Presenting these automatically generated sub-threshold modulation programs to the user allows the user to program the IPG 14 efficiently and intelligently and eventually arrive at an optimal sub-threshold modulation program (or programs) or sub-threshold therapy regimen for the patient.

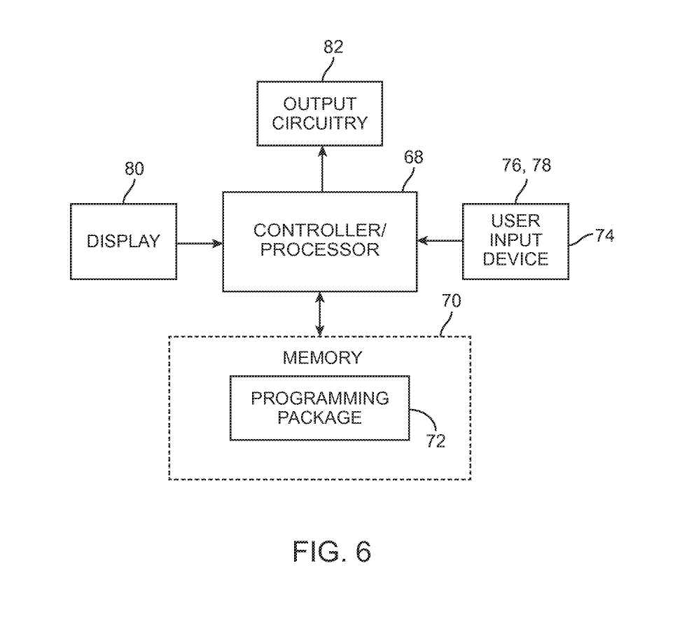

In practice, the user performs these programming sessions of the IPG 14 on the CP 18. As shown in FIG. 6, the overall appearance of the CP 18 is that of a laptop personal computer (PC), and in fact, may be implemented using a PC that has been appropriately configured to include a directional-programming device and programmed to perform the functions described herein. Alternatively, the CP 18 may take the form of a mini-computer, personal digital assistant (PDA), etc., or even a remote control (RC) with expanded functionality. Thus, the programming methodologies can be performed by executing software instructions contained within the CP 18. Alternatively, such programming methodologies can be performed using firmware or hardware. In any event, the CP 18 may actively control the characteristics of the electrical modulation generated by the IPG 14 to allow the optimum modulation parameters to be determined based on patient feedback and for subsequently programming the IPG 14 with the optimum modulation parameter.

To allow the user to perform these functions, the CP 18 includes a user input device (e.g., a mouse 76 and a keyboard 78), and a programming display screen 80 housed in a case 82. It is to be understood that in addition to, or in lieu of, the mouse 76, other directional programming devices may be used, such as a trackball, touchpad, joystick, or directional keys included as part of the keys associated with the keyboard 78.

In the illustrated embodiment described below, the display screen 80 takes the form of a conventional screen, in which case, a virtual pointing device, such as a cursor controlled by a mouse, joy stick, trackball, etc., can be used to manipulate graphical objects on the display screen 80. In alternative embodiments, the display screen 80 takes the form of a digitizer touch screen, which may either passive or active. Further details discussing the use of a digitizer screen for programming are set forth in U.S. Provisional Patent Application Ser. No. 61/561,760, entitled “Technique for Linking Electrodes Together during Programming of Neurostimulation System,” which is expressly incorporated herein by reference.

Referring now to FIG. 6, the CP 18 includes a controller/processor 68 (e.g., a central processor unit (CPU)) and memory 70 that stores a programming package 72, which can be executed by the controller/processor 68 to allow the user to program the IPG 14 and RC 16 and the plurality of calibrated sub-threshold modulation programs. Significant to the present inventions, the controller/processor 68 is configured for automatically generating, based on the selected super-threshold modulation program, the plurality of sub-threshold modulation programs that may be used on the patient.

In addition, the CP 18 further includes a user input device 74 (such as the mouse 76 or the keyboard 78 described above) to provide user commands. Notably, while the controller/processor 68 is shown in FIG. 6 as a single device, the processing functions and controlling functions can be performed by a separate controller and processor. Thus, it can be appreciated that the controlling functions described below as being performed by the CP 18 can be performed by a controller, and the processing functions described below as being performed by the CP 18 can be performed by the microcontroller 48 of the IPG 14 or the processor of the RC 16.

Execution of the programming package 72 by the controller/processor 68 provides a multitude of display screens (not shown) that can be navigated through via use of the mouse 76. These display screens allow the clinician to, among other functions, to select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), define the configuration and orientation of the leads, initiate and control the electrical modulation energy output by the neuromodulation leads 12, and select and program the IPG 14 with modulation parameters in both a surgical setting and a clinical setting. Further details discussing the above-described CP functions are disclosed in U.S. patent application Ser. No. 12/501,282, entitled “System and Method for Converting Tissue Stimulation Programs in a Format Usable by an Electrical Current Steering Navigator,” and U.S. patent application Ser. No. 12/614,942, entitled “System and Method for Determining Appropriate Steering Tables for Distributing Modulation energy Among Multiple Neuromodulation Electrodes,” which are expressly incorporated herein by reference. Execution of the programming package 72 provides a user interface that conveniently allows a user to program the IPG 14.

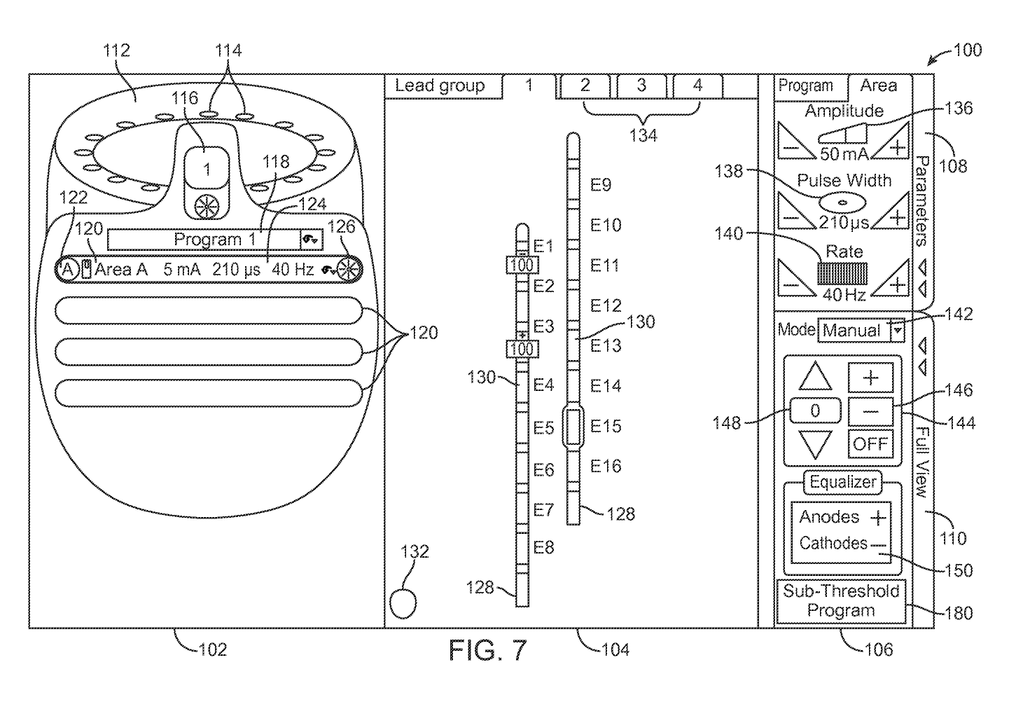

Referring now to FIG. 7, a programming screen 100 that can be generated by the CP 18 to allow a user to program the IPG 14 will be described. In the illustrated embodiment, the programming screen 100 comprises three panels: a program selection panel 102, a lead display panel 104, and a modulation parameter adjustment panel 106. Some embodiments of the programming screen 100 may allow for closing and expanding one or both of the lead display panel 102 and the parameter adjustment panel 106 by clicking on the tab 108 (to show or hide the parameter adjustment panel 106) or the tab 110 (to show or hide the full view of both the lead selection panel 104 and the parameter adjustment panel 106).

The program selection panel 102 provides information about modulation programs and coverage areas that have been, or may be, defined for the IPG 14. In particular, the program selection panel 102 includes a carousel 112 on which a plurality of modulation programs 114 (in this case, up to sixteen) may be displayed and selected. The program selection panel 102 further includes a selected program status field 116 indicating the number of the modulation program 114 that is currently selected (any number from “1” to “16”). In the illustrated embodiment, program 1 is the only one currently selected, as indicated by the number “1” in the field 116. The program selection panel 102 further comprises a name field 118 in which a user may associate a unique name to the currently selected modulation program 114.

The program selection panel 102 further comprises a plurality of coverage areas 120 (in this case, up to four) with which a plurality of modulation parameter sets can respectively be associated to create the currently selected modulation program 114 (in this case, program 1). Each coverage area 120 that has been defined includes a designation field 122 (one of letters “A”-“D”), and an electrical pulse parameter field 124 displaying the electrical pulse parameters, and specifically, the pulse amplitude, pulse width, and pulse rate, of the modulation parameter set associated with the that coverage area. In this example, only coverage area A is defined for program 1, as indicated by the “A” in the designation field 122. The electrical pulse parameter field 124 indicates that a pulse amplitude of 5 mA, a pulse width of 210 μs, and a pulse rate of 40 Hz has been associated with coverage area A.

Each of the defined coverage areas 120 also includes a selection icon 126 that can be alternately actuated to activate or deactivate the respective coverage area 120. When a coverage area is activated, an electrical pulse train is delivered from the IPG 14 to the electrode array 26 in accordance with the modulation parameter set associated with that coverage area. Notably, multiple ones of the coverage areas 120 can be simultaneously activated by actuating the selection icons 126 for the respective coverage areas. In this case, multiple electrical pulse trains are concurrently delivered from the IPG 14 to the electrode array 26 during timing channels in an interleaved fashion in accordance with the respective modulation parameter sets associated with the coverage areas 120. Thus, each coverage area 120 corresponds to a timing channel.

To the extent that any of the coverage areas 120 have not been defined (in this case, three have not been defined), they include text “click to add another program area”), indicating that any of these remaining coverage areas 120 can be selected for association with a modulation parameter set. Once selected, the coverage area 120 will be populated with the designation field 122, electrical pulse parameter field 124, and selection icon 126.

The parameter adjustment panel 106 includes a pulse amplitude adjustment control 136 (expressed in milliamperes (mA)), a pulse width adjustment control 138 (expressed in microseconds (μs)), and a pulse rate adjustment control 140 (expressed in Hertz (Hz)), which are displayed and actuatable in all the programming modes. Each of the controls 136-140 includes a first arrow that can be actuated to decrease the value of the respective modulation parameter and a second arrow that can be actuated to increase the value of the respective modulation parameter. Each of the controls 136-140 also includes a display area for displaying the currently selected parameter. In response to the adjustment of any of electrical pulse parameters via manipulation of the graphical controls in the parameter adjustment panel 106, the controller/processor 68 generates a corresponding modulation parameter set (with a new pulse amplitude, new pulse width, or new pulse rate) and transmits it to the IPG 14 via the telemetry circuitry 54 for use in delivering the modulation energy to the electrodes 26.

The parameter adjustment panel 106 includes a pull-down programming mode field 142 that allows the user to switch between a manual programming mode, an electronic trolling programming mode, and a navigation programming mode. Each of these programming modes allows a user to define a modulation parameter set for the currently selected coverage area 120 of the currently selected program 114 via manipulation of graphical controls in the parameter adjustment panel 106 described above, as well as the various graphical controls described below.

The manual programming mode is designed to allow the user to manually define the fractionalized electrical current for the electrode array with maximum flexibility; the electronic trolling programming mode is designed to quickly sweep the electrode array using a limited number of electrode configurations to gradually steer an electrical field relative to the neuromodulation leads until the targeted modulation site is located; and the navigation programming mode is designed to sweep the electrode array using a wide number of electrode configurations to shape the electrical field, thereby fine tuning and optimization the modulation coverage for patient comfort.

As shown in FIG. 7, the manual programming mode has been selected. In the manual programming mode, each of the electrodes 130 of the graphical leads 128, as well as the graphical case 132, may be individually selected, allowing the clinician to set the polarity (cathode or anode) and the magnitude of the current (percentage) allocated to that electrode 130, 132 using graphical controls located in an amplitude/polarity area 144 of the parameter adjustment panel 106.

In particular, a graphical polarity control 146 located in the amplitude/polarity area 144 includes a “+” icon, a “−” icon, and an “OFF” icon, which can be respectively actuated to toggle the selected electrode 130, 132 between a positive polarization (anode), a negative polarization (cathode), and an off-state. An amplitude control 148 in the amplitude/polarity area 144 includes an arrow that can be actuated to decrease the magnitude of the fractionalized current of the selected electrode 130, 132, and an arrow that can be actuated to increase the magnitude of the fractionalized current of the selected electrode 130, 132. The amplitude control 148 also includes a display area that indicates the adjusted magnitude of the fractionalized current for the selected electrode 134. The amplitude control 148 is preferably disabled if no electrode is visible and selected in the lead display panel 104. In response to the adjustment of fractionalized electrode combination via manipulation of the graphical controls in the amplitude/polarity area 144, the controller/processor 68 generates a corresponding modulation parameter set (with a new fractionalized electrode combination) and transmits it to the IPG 14 via the telemetry circuitry 54 for use in delivering the modulation energy to the electrodes 26.

In the illustrated embodiment, electrode E1 has been selected as a cathode and electrode E3 has been selected as anode with 100% of the cathodic and anodic current allocated to each of them respectively. Although the graphical controls located in the amplitude/polarity area 144 can be manipulated for any of the electrodes, a dedicated graphical control for selecting the polarity and fractionalized current value can be associated with each of the electrodes, as described in U.S. Patent Publication No. 2012/0290041, entitled “Neurostimulation System with On-Effector Programmer Control,” which is expressly incorporated herein by reference.

The parameter adjustment panel 106, when the manual programming mode is selected, also includes an equalization control 150 that can be actuated to automatically equalize current allocation to all electrodes of a polarity selected by respective “Anode +” and “Cathode −” icons.

Significant to the present inventions, the parameter adjustment panel 106 also comprises a sub-threshold modulation program control 180 that can be actuated to automatically generate sub-threshold modulation programs based on a selected super-threshold modulation program. In the illustrated embodiment, the selected super-threshold modulation program is “Program 1” as shown in the program selection panel 102, wherein super-threshold electrical energy is delivered through electrodes E1 and E3 as shown in the lead display panel 104, with modulation parameters of 5 mA pulse amplitude, 210 μs pulse width and 40 Hz pulse rate.

The super-threshold modulation program may be selected based on the patient's individual needs and targeted area for neuromodulation therapy. In practice, the user typically selects a super-threshold modulation program that is believed to be effective (e.g., the patient may feel paresthesia in the targeted therapy area in response to the super-threshold modulation program).

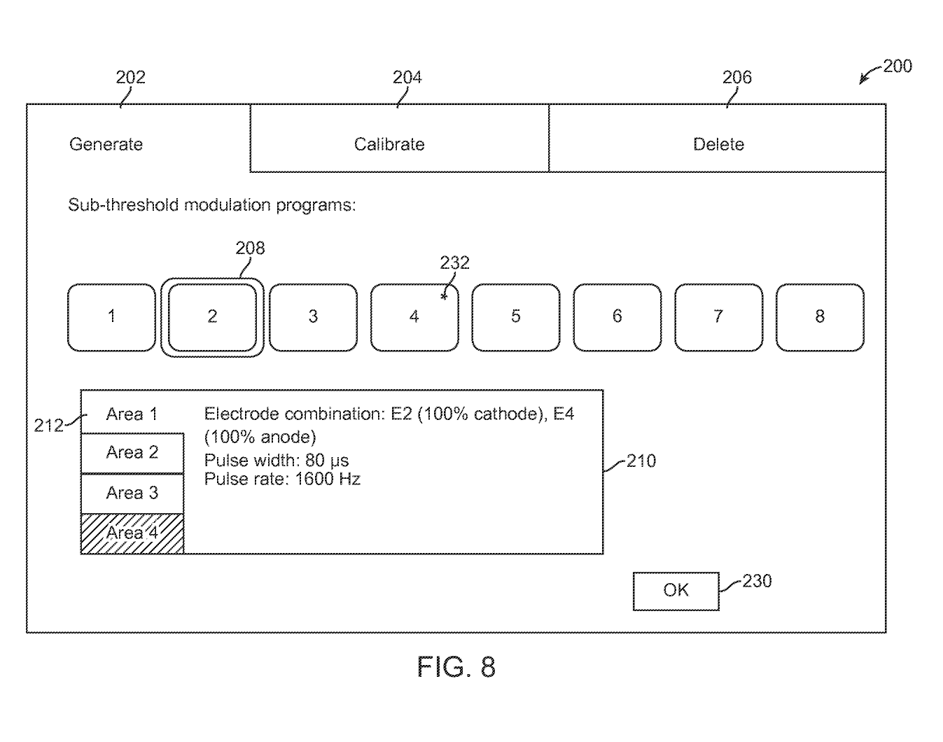

When the sub-threshold modulation program control 180 is actuated, the user is automatically taken to a sub-threshold modulation program screen 200 as shown in FIG. 8. In the illustrated embodiment, the “generate” tab 202 has been opened, and eight sub-threshold modulation programs have been created based on the selected super-threshold modulation program. It should be appreciated that other embodiments of the sub-threshold modulation screen 200 may generate fewer or greater number of sub-threshold modulation programs. Each sub-threshold modulation program is different from the other, and all of them are automatically generated based on an algorithm in the CP 18 that modifies the modulation parameters of the selected super-threshold modulation program.

In the illustrated embodiment, “Program 2” is shown as being selected by the graphical control 208. When a particular sub-threshold modulation program is selected, a program details box 210 is automatically populated showing details of the automatically generated program. As can be seen in the program details box 210, the modulation parameters of the automatically generated sub-threshold modulation programs are slightly varied from the original parameters of the super-threshold modulation program or include modulation parameter sets that are especially beneficial for sub-threshold modulation therapy as will be described further below. The modulation parameters include electrode combinations (or fractionalized electrode combinations), polarity of the electrodes, burst rate, pulse shape, pulse rate and pulse width.

For example, in “Program 2,” the electrode combination of the super-threshold modulation program selected in the manual programming screen 100 is switched such that electrode E2 is now configured as the cathode and electrode E4 is configured as the anode instead of electrodes E1 and E3, keeping the pulse width and pulse rate at the sub-threshold levels of 80 μs and 1600 Hz respectively. In making a small change such as this, substantially same or similar nerve fibers are stimulated, yet giving the user a chance to assess if this modified electrode configuration works effectively for sub-threshold modulation therapy.

In another one of the automatically generated sub-threshold modulation programs (not illustrated), only the pulse width and pulse rate are modified to the sub-threshold levels. In another example (not illustrated), the polarity of the electrodes of the selected super-threshold modulation program may be switched such that electrode E1 is now configured as an anode and electrode E3 is now configured as a cathode, while keeping the pulse width and pulse rate at sub-threshold levels.

In yet another one of the automatically generated sub-threshold modulation programs (not illustrated), the burst rate may be modified such that the inter-burst quiescent period is 3 ms and the intra-burst interval is 5 ms, but keeping the existing electrode combination of the selected super-threshold modulation program in the manual programming screen 100 and pulse rate and pulse width adjusted to sub-threshold levels. It has been observed that burst stimulation, wherein each of the sub-threshold modulation programs defines an inter-burst quiescent period of at least 1 ms and less than 5 seconds, and an intra-burst interval in the range of 0.1 ms to 10 ms, is especially effective in sub-threshold modulation therapy.

Although the foregoing examples have focused on modifying a single modulation parameter, it should be appreciated that each sub-threshold modulation program comprises a plurality of modulation parameter sets and any or all of them may be modified.

It should also be appreciated that each sub-threshold modulation program may have multiple timing channels corresponding to different areas of the patient all of which may be derived from the selected super-threshold modulation program. In the illustrated embodiment, each sub-threshold modulation program can have up to four coverage areas 212 that may be simultaneously stimulated as shown in the program details tab 210. Similarly, the user can dick other areas 212 to see the program details for that particular area. In the illustrated embodiment, “Program 2” only defines modulation parameters for three areas, so “Area 4” is checked off to denote that it is not selectable by the user. Similarly, the user can view details of the other generated sub-threshold modulation programs using graphical control 208.

Thus, it can be appreciated that there are infinite combinations of modulation parameter sets that may be generated based on a single selected super-threshold modulation program. Keeping the sub-threshold modulation programs substantially similar to the selected super-threshold modulation programs affords the user the opportunity to experiment with various combinations and permutations of the modulation parameters of the selected super-threshold modulation program while maintaining efficacy of the treatment.

It should be appreciated that the pulse amplitude of the automatically generated sub-threshold modulation programs remains zero until each sub-threshold modulation program is calibrated. Calibrating the sub-threshold modulation programs entails determining the perception threshold (i.e., the amplitude at which the patient first perceives paresthesia in response to the delivered electrical energy). Calibration is an important step because the perception threshold for all programs may not be the same, and may be based on the particular modulation parameters of the program. Thus, calibrating each program independently ensures that the sub-threshold amplitude is appropriate for each sub-threshold modulation program.

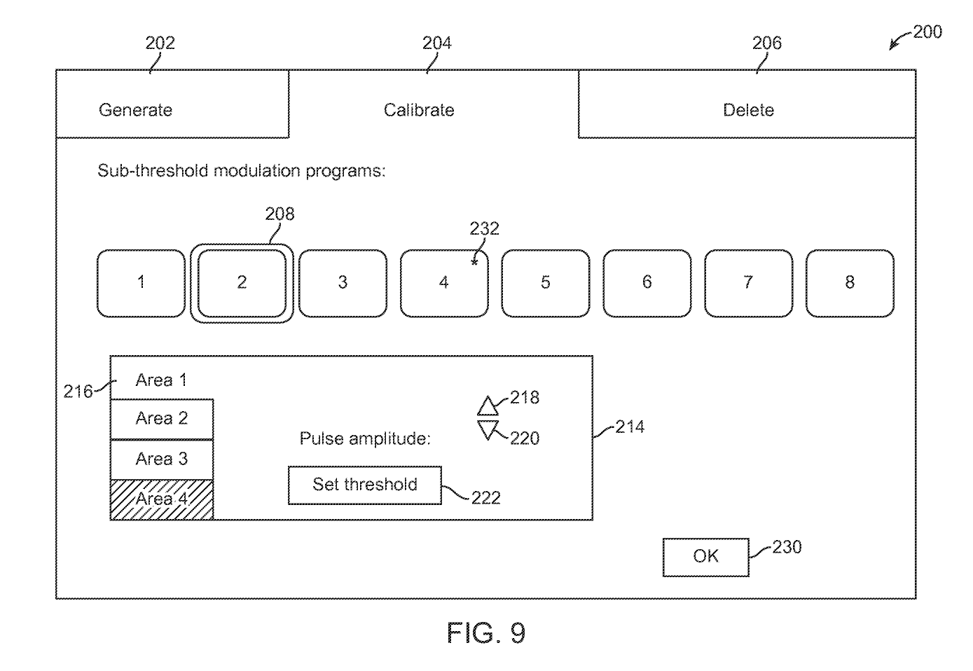

Referring now to FIG. 9, the “calibrate” tab 204 of the sub-threshold modulation program screen 200 has been opened. Once again, the calibrate tab 204 also shows all eight generated sub-threshold modulation programs and “Program 2” is again shown as being selected using graphical control 208. When a particular sub-threshold modulation program is selected, a calibration box 214 is automatically populated. The calibration box 214 allows the user to determine the perception threshold for each area of the program. As was the case with the program details tab 210, the calibration tab 214 also has up to four area tabs 216, each of which has to be individually calibrated.

To calibrate “Area 1” of “Program 2”, the pulse amplitude is incrementally increased using graphical control 218. The user keeps increasing the amplitude until the patient reports a feeling of paresthesia, at which point, the “Set Threshold” control 222 can be actuated such that that particular amplitude value at which paresthesia was first perceived (perception threshold) is automatically recorded.

After the sub-threshold modulation program has been calibrated, the CP 18 is configured to automatically calculate the sub-threshold amplitude for that sub-threshold modulation program based on the perception threshold. Since the goal of sub-threshold modulation therapy is to provide therapy without inducing paresthesia, the sub-threshold amplitude is purposely lower than the perception threshold, and is typically calculated as a function (e.g., percentage) of the perception threshold. For example, the sub-threshold amplitude may be 70% of the perception threshold. Or, in another example, the sub-threshold amplitude may be 50% of the perception threshold.

Similarly, the user can dick other areas 216 to calibrate the other areas of “Program 2” as well. Or the user may select other programs to calibrate using graphical control 208. Sub-threshold modulation programs that have already been calibrated may be denoted by a symbol 232, such as the one shown for “Program 4” in FIG. 9.

Once the sub-threshold modulation program has been calibrated, it is then saved into the memory. In a preferred embodiment, the calibrated sub-threshold modulation programs may be additionally stored into the RC 16 such that the patient can maintain some control over the programming of the IPG 14 at home. For example, if the patient likes a particular program and/or wants to skip over another one, he may easily do so with the RC 16. The patient may also be able to modify the pulse amplitude of a particular sub-threshold modulation program.

To this end, the user may further define (not illustrated), a minimum amplitude level and a maximum amplitude level such that the patient, at his/her own discretion, is able to adjust the pulse amplitude within a range. The range is typically determined based on the perception threshold. For example, assuming that the sub-threshold amplitude level is set to be 50% of the perception threshold, the minimum amplitude level may be defined as 30% of the perception threshold, and the maximum amplitude may be defined as 70% of the perception threshold. Or, in another example, if the user wants therapy to remain at a tighter range, the minimum amplitude may be set at 40% of the perception threshold and the maximum amplitude may be set at 60% of the perception threshold.

It should be appreciated that any or all of the automatically generated sub-threshold modulation programs may be deleted based on user discretion (before or after calibration). This allows the user more control in deciding the sub-threshold modulation therapy that is ideal for the patient. The deleted programs may be replaced with new sub-threshold modulation programs, as will be described further below.

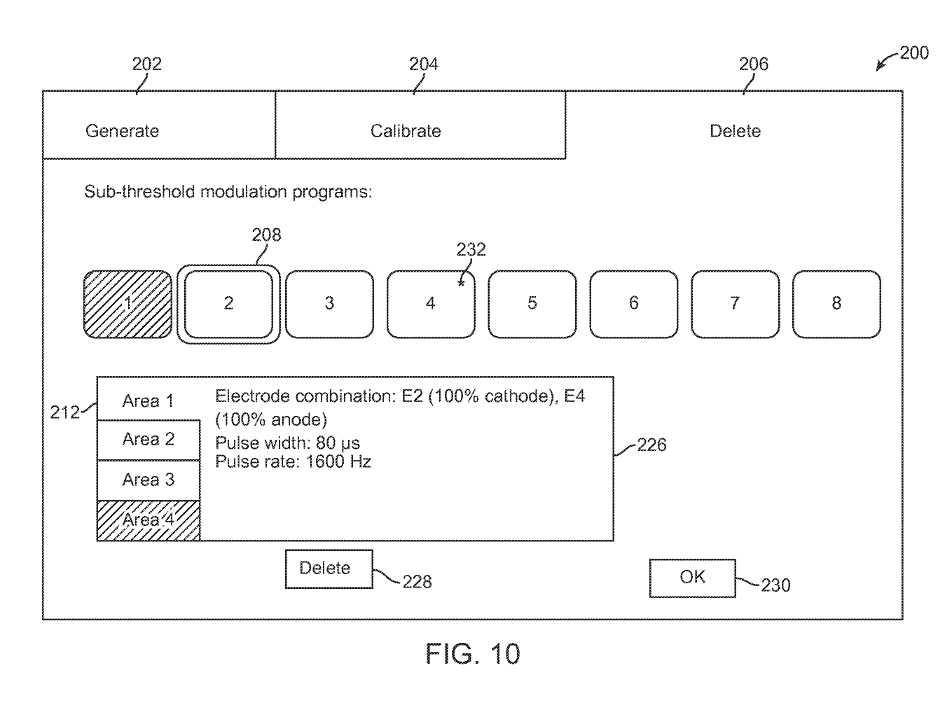

Referring now to FIG. 10, the “delete” tab 206 of the sub-threshold modulation program screen 200 has been opened. The delete tab 206 enables the user to delete any of the automatically generated sub-threshold modulation programs using the graphical control 208. When a particular sub-threshold modulation program is selected, a deletion box 226 is automatically generated that allows the user to review the program details of that particular sub-threshold modulation program, and delete the program using the graphical “Delete” button 228. As shown in the illustrated embodiment, “Program 1” has been deleted as denoted by the check marks on “Program 1” and is therefore not selectable by the user.

It should be appreciated that if the user deletes a particular sub-threshold modulation program, but wants to try out a different automatically generated sub-threshold modulation program based on another super-threshold modulation program, the CP 18 automatically generates a new sub-threshold modulation program based on the other super-threshold modulation program in place of the deleted sub-threshold modulation program.

In particular, to select a new super-threshold modulation program, the user may click the graphical “OK” button 230 to be taken back to the manual programming screen 100 shown in FIG. 7. The user may then select a new super-threshold modulation program on the manual programming screen 100 and once again select the sub-threshold modulation program control 180 to be taken back to the sub-threshold modulation program screen 200. This time, however, new sub-threshold modulation programs will be generated based on the new super-threshold modulation program selected by the user and the new sub-threshold modulation programs will be displayed in place of previously deleted sub-threshold modulation programs. For example, assuming that both “Program 1” and “Program 2” are deleted in FIG. 10, and the user selects a different super-threshold modulation program from which to generate new sub-threshold modulation programs, “Program 1” and “Program 2” will no longer be shown as deleted, but will rather display details of the new sub-threshold modulation programs that have been generated based on the different super-threshold modulation program selected by the user.

Once the relevant sub-threshold modulations programs have been deleted, and all the desired sub-threshold modulation programs have been calibrated and saved onto the memory, the plurality of sub-threshold modulation programs may then be used on the patient. This is typically the end of the programming session, although the user will be able to monitor and analyze the efficacy of the sub-threshold modulation programs that are being tested out on the patient. As mentioned before, the patient is able to maintain some control over the sub-threshold therapy using the RC 16.

In particular, the plurality of sub-threshold modulation programs are typically repeatedly cycled through based on a predetermined time schedule such that electrical energy delivered in accordance to a first sub-threshold modulation program is seamlessly replaced by electrical energy in accordance to a second sub-threshold modulation program. Advantageously, since they follow the predetermined time schedule, the therapy provided by any of these different sub-threshold modulation programs may be analyzed to determine which of the programs is most efficacious for the patient. In the preferred embodiment, the patient may be able to provide feedback on the efficacy of the sub-threshold modulation programs, through the RC 16, which may then be recorded and viewed later by the user at another programming session.

In an alternate embodiment, the SCM system 10 may detect a physiological parameter (e.g., patient activity level, patient posture, etc.), to estimate the efficacy of a particular sub-threshold modulation program. For example, it can be assumed that the level of physical activity of a patient is inversely proportional to the pain level experienced by the patient (i.e., if the patient is awake and physically active, this indicates that current modulation parameter set is efficacious, whereas if the patient is excessively asleep or otherwise in the prone position, this indicates that the current modulation parameter set is not efficacious). In one technique, the physical activity level of the patient is estimated from the magnitude of time varying electrical parameter data measured from the electrodes 26 or data measured from other sensors (impedance, activity, accelerometer, etc.), as described in U.S. patent application Ser. No. 12/024,947, entitled “Neurostimulation System and Method for Measuring Patient Activity,” which is expressly incorporated herein by reference. In another technique, the physical activity level of the patient is estimated from a frequency that an orientation sensitive component implanted within the patient detects a change in orientation, as described in U.S. patent application Ser. No. 13/446,191, entitled “Sensing Device for Indicating Posture of Patient Implanted with a Neurostimulation Device, which is expressly incorporated herein by reference.

Thus, by repeatedly cycling through the plurality of automatically generated sub-threshold modulation programs, and estimating the efficacy of each program in the ways described above, different modulation parameter sets can be experimented with and assessed, thereby making the process of finding an optimal sub-threshold modulation regimen for the patient easier and more efficient. Further details on cycling through sub-threshold modulation programs are disclosed in U.S. Patent Application Ser. No. 61/832,088 (Attorney Docket No. 13-0121PV01) entitled “System and method for delivering modulated sub-threshold therapy to a patient,” which is expressly incorporated herein by reference.

Although the illustrated embodiments have focused on using the manual programming mode to select the super-threshold modulation program that is used to automatically generate the sub-threshold modulation programs, it should be appreciated that any of the other programming modes of the CP 18 may also be similarly used.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. A method of providing sub-threshold modulation therapy to a patient, comprising:
receiving input from a user;
generating a super-threshold modulation program based on the received user input;
delivering super-threshold modulation energy to the patient in accordance with the super-threshold modulation program;
automatically deriving a plurality of different sub-threshold modulation programs from the super-threshold modulation program;
storing the plurality of sub-threshold modulation programs in memory; and
delivering sub-threshold modulation energy to the patient in accordance with at least one of the sub-threshold modulation programs, thereby providing therapy to the patient.

2. The method of claim 1, wherein each of the super-threshold modulation program and the plurality of sub-threshold modulation programs comprises a plurality of modulation parameter sets respectively corresponding to different areas of the patient.

3. The method of claim 1, further comprising:
receiving additional input from the user;
selecting, based on the additional user-input, one of the plurality of sub-threshold modulation programs;
determining a perception threshold of the selected sub-threshold modulation program;
determining a sub-threshold amplitude value for the selected sub-threshold modulation program as a function of the perception threshold to calibrate the selected sub-threshold modulation program; and
storing the calibrated sub-threshold modulation program in the memory.

4. The method of claim 3, wherein the function is a percentage of the perception threshold.

5. The method of claim 4, wherein the percentage is in the range of 30%-70%.

6. The method of claim 3, further comprising saving the calibrated sub-threshold modulation program onto a remote programming device.

7. The method of claim 6, further comprising:
receiving further additional input from the user; and
defining a minimum amplitude level and a maximum amplitude level for the saved sub-threshold modulation program based on the received further additional user input.

8. The method of claim 1, further comprising
repeatedly cycling through the plurality of sub-threshold modulation programs to deliver the sub-threshold modulation energy to the patient.

9. The method of claim 1, further comprising:
receiving additional input from the user; and
deleting one of the sub-threshold modulation programs from the memory in response to the additional user-input.

10. The method of claim 9, further comprising:
receiving further additional input from the user;
generating another super-threshold modulation program based on the received further additional user-input;
delivering super-threshold modulation energy to the patient in accordance with the other super-threshold modulation program;
automatically deriving another sub-threshold modulation program from the other super-threshold modulation program; and
storing the other sub-threshold modulation program in memory in place of the deleted sub-threshold modulation program.

11. The method of claim 1, wherein each of the plurality of sub-threshold programs defines a pulse width less than 100 μs.

12. The method of claim 1, wherein each of the plurality of sub-threshold programs defines a pulse rate greater than 1500 Hz.

13. The method of claim 1, wherein each of the plurality of sub-threshold programs defines an inter-burst quiescent period of at least 1 ms and less than 5 seconds, and an intra-burst interval in the range of 0.1 msec to 10 msec.

14. A neuromodulation system, comprising:
a plurality of electrical terminals configured for being respectively coupled to a plurality of electrodes;
modulation output circuitry configured for delivering modulation energy to the electrical terminals;
a user interface for receiving input from a user; and
control/processing circuitry configured for generating a super-threshold modulation program based on the received user-input, controlling the modulation output circuitry to deliver super-threshold modulation energy in accordance with the superthreshold modulation program, automatically deriving a plurality of different subthreshold modulation programs from the super-threshold modulation program, and controlling the modulation output circuitry to deliver sub-threshold modulation energy to a patient in accordance with at least one of the sub-threshold modulation programs.

15. The neuromodulation system of claim 14, further comprising memory configured for storing at least one of the super-threshold modulation program and the plurality of sub-threshold modulation programs.

16. The neuromodulation system of claim 14, wherein each of the superthreshold modulation program and the plurality of sub-threshold modulation programs comprises a plurality of modulation parameter sets respectively corresponding to different areas of the patient.

17. The neuromodulation system of claim 14, wherein the user interface is configured for receiving additional input from the user, and wherein the control/processing circuitry is further configured for selecting, based on the additional user-input, one of the plurality of sub-threshold modulation programs.

18. An external control device for programming an implantable neuromodulator coupled to an electrode array, comprising:
a user interface including control elements for receiving input from a user;
telemetry circuitry configured for communicating with the neuromodulator; and
control/processing circuitry configured for generating a super-threshold modulation program based on the received user-input, directing the neuromodulator to deliver super-threshold modulation energy in accordance with the super-threshold modulation program, automatically deriving a plurality of different sub-threshold modulation programs from the super-threshold modulation program, and directing the neuromodulator to deliver sub-threshold modulation energy to a patient in accordance with at least one of the sub-threshold modulation programs.

19. The external control device of claim 18, further comprising memory configured for storing at least one of the super-threshold modulation program and the plurality of sub-threshold modulation programs.

20. The external control device of claim 18, wherein each of the super-threshold modulation program and the plurality of sub-threshold modulation programs comprises a plurality of modulation parameter sets respectively corresponding to different areas of the patient.
